(19) United States
(12) Patent Application Publication
(10) Pub. No.: US 2025/0268681 A1
(43) Pub. Date: Aug. 28, 2025

(54) PERINEAL ACCESS TOOL

(71) Applicant: CLEAR GUIDE MEDICAL, INC., Baltimore, MD (US)

(72) Inventors: David Woodrum, Rochester, MN (US); Purnima Rajan, Baltimore, MD (US); Pezhman Foroughi, Baltimore, MD (US); Alican Demir, Baltimore, MD (US); Paul C. Clark, Baltimore, MD (US)

(73) Assignee: Clear Guide Medical, Inc., Baltimore, MD (US)

(21) Appl. No.: 18/856,850

(22) PCT Filed: Apr. 14, 2023
(86) PCT No.: PCT/US2023/018730

§ 371 Date: Oct. 14, 2024

Related U.S. Application Data
(60) Provisional application No. 63/331,615, filed on Apr. 15, 2022.

(51) Int. Cl. A61B 90/11; A61B 90/00; A61B 90/14; A61B 90/30

(52) U.S. Cl. CPC A61B 90/11; A61B 90/14; A61B 90/30; A61B 90/39; A61B 2090/309; A61B 2090/3995

(57) ABSTRACT

A perineal access tool is intended to facilitate trans perineal prostate procedures within the MRI suite. The tool facilitates instrument guidance in connection with interventions, e.g., prostate interventions. Multi-modal markers allow the tool to be registered during the imaging procedures. The tool includes a guidance assembly that allows a user to guide an intervention instrument directly to the target area when performing image guided intervention procedures.

### TECHNICAL FIELD

The present disclosure relates to devices used in transperineally procedures.

### BACKGROUND ART

Prostate cancer (PCa) is the most common cancer in men and is the second leading cause of cancer death in the United States. It is estimated that about 1 man in 41 will die of PCa. The American Cancer Society estimates that in 2022 there will be about 268,490 new cases of PCa diagnosed in the U.S., alone, and about 34,500 deaths attributable to Pca. Alarmingly, about 1 man in 8 will be diagnosed with prostate cancer during his lifetime.

There are the two principal approaches for the diagnosis of PCa: the trans perineal (TP) biopsy and the transrectal (TR) biopsy. The systematic TR prostate biopsy, which is the gold standard for the detection of PCa, has been conducted for decades worldwide. This method, however, reportedly underestimates PCa incidence with a false negative rate up to 49%. Additionally, TR prostate biopsy has been reported to cause severe complications such as rectal bleeding, fever, sepsis, hematuria, and acute urinary retention. Due to the high false negative and complication rates of the systematic TR prostate biopsy, the TP approach was introduced to improve the detection rate and safety of prostate biopsy.

The TP approach includes obtaining tissue or cell specimens by passing one or more biopsy needles through the perineum and into the prostate. The first technique uses a device called a stepper, which is used to cradle the ultrasound probe and provide a guidance grid for biopsy needle insertion. Urologists who have performed brachytherapy for prostate cancer would be familiar with this device. The downside of this approach is that each biopsy of the prostate requires a skin puncture, which limits the utility of this approach in the clinic where only local anesthesia is used. This technique may still be used, but an excellent skin block is paramount.”

Some urologists also employ a freehand approach, using a needle access guide. In this approach, optimally, only one or two skin punctures are needed for each side of the prostate. The biopsy needle may be reintroduced for multiple biopsies without repeat puncture of the skin through a needle access guide placed through the anesthetized skin of the perineum. The biopsy needles may be guided by an ultrasound probe in the rectum. This approach allows for improved patient comfort in the clinic setting.

### DISCLOSURE OF INVENTION

In accordance with an embodiment of the invention, a perineal access device is provided including a base, first and second leg separators removably secured to the base and a guidance assembly mounted to the base and disposed between the first and second leg separators. The guidance assembly includes an access frame having an upper window configured to provide access to a patient's perinium and a lower window configured to provide access to the patient's rectum. The access frame is supported by first and second frame legs. A top platter extends from the access frame and forms a platter angle with the access frame. A guidance plate is disposed within the access frame. The guidance plate includes a grid having one or more channels, and first and second multi-modal markers attached to first and second frame legs, respectively. An optical head is attached to one of the first and second leg separators and positioned at a pitch angle to provide a maximum field of view for said optical head.

In accordance with another embodiment of the invention, a perineal access device is provided including a base, first and second leg separators removably secured to the base and a guidance assembly mounted to the based and disposed between said first and second leg separators. The guidance assembly includes an access frame including an upper window configured to provide access to a patient's perinium and a lower window configured to provide access to a patient's rectum. The perineal access device further includes an optical head attached to one of the first and second leg separators and positioned at a pitch angle to provide a maximum field of view for the optical head.

In accordance with yet another embodiment of the invention, a guidance assembly for a perineal access device includes an access frame having an upper window configured to provide access to a patient's perinium and a lower window configured to provide access to the patient's rectum. The access frame is supported by first and second frame legs. The guidance assembly has a top platter extending from the access frame, where the top platter forms a platter angle with the access frame. A guidance plate is disposed within the access frame, and the guidance plate has a grid having one or more channels. First and second multi-modal markers are attached to the first and second frame legs, respectively.

### BRIEF DESCRIPTION OF DRAWINGS

FIG. 1 shows a perineal access tool in accordance with an embodiment of the invention.

FIG. 2 illustrates an exploded view of the perineal access tool of FIG. 1.

FIG. 3 depicts a side view of a guidance assembly in accordance with an embodiment of the invention.

FIG. 4 shows a front view of a guidance assembly in accordance with an embodiment of the invention.

FIG. 5 depicts a close-up view of an optical head in accordance with an embodiment of the invention.

FIG. 6 illustrates a rear perspective view of a grid plate in accordance with the invention.

FIG. 7 depicts a front perspective view the grid plate of FIG. 6

FIG. 8 depicts a cross sectional view of the grid plate of FIG. 6.

FIG. 9 shows a front perspective view grid plate in accordance with an embodiment of the invention.

FIG. 10 illustrates a front view of the grid plate of FIG. 9.

FIG. 11 shows a front view of a grid plate in accordance with still another embodiment of the invention.

FIG. 12 depicts a base of the grid plate of FIG. 11.

FIG. 13 shows a pivot element of the grid plate of FIG. 11.

### DESCRIPTION OF EMBODIMENTS

The present disclosure describes devices and methods that allow operators to perform navigation-assisted prostate interventions using certain types of intervention instruments, such as needle-like instruments using visual or automatic termination criteria. Example applications may include needle biopsies, tumor ablations, catheter insertion, and other instruments, all of which may use several types of image data. Typically, these instruments are inserted into a patient's body at very specific locations, orientations, and depths to reach predetermined target areas, where they perform an instrument-specific action or function, which may include tissue sampling, heating, cooling, liquid deposition, suction, or serving as a channel for other objects. More particularly, the present disclosure is directed to a perineal access device that facilitates operator access to perform navigation-assisted biopsy, ablation and other interventional prostate procedures.

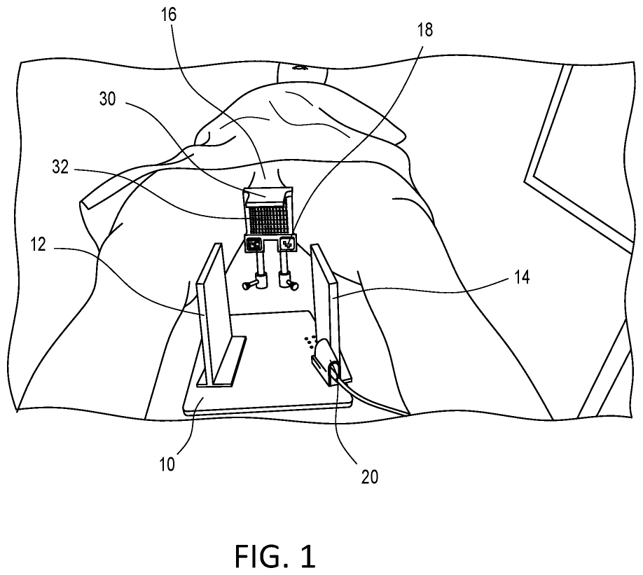

FIG. 1 illustrates a perineal access tool in accordance with an embodiment of the invention deployed with a patient. A base 10 is provided upon which first and second leg separators 12 and 14 may be disposed. A guidance assembly 16 is connected to base 10 and positioned upstream from first and second leg separators 12 and 14. Guidance assembly 16 is positioned to abut the crotch area of the patient when the patient is laid supine on base 10. In some embodiments, guidance assembly 16 may be provided with multi-modal markers 18 to facilitate image registration. An optical head 20 including a light source and a camera is attached to one of first and second leg separators 12 and 14.

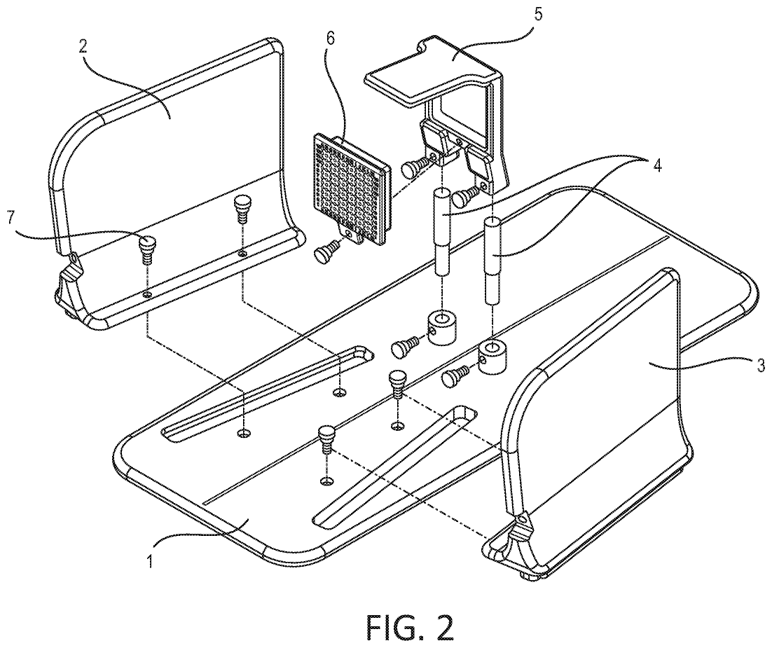

As illustrated in FIG. 2, in at least one embodiment guidance assembly 16 includes an access frame 22 supported by first and second frame legs 24 and 26 each having an interior post receptacle or longitudinal channel. Access frame 22 includes an upper window 50 configured to provide access to the perinium and a lower window 52 configured to provide access to the rectum. Access frame 22 may be attached to base 10 in a manner that allows for height adjustment. In some embodiments, frame legs 24 and 26 may be secured to base 10 via height adjustment posts 28 which may be threadedly engaged with the interior post receptacles in a manner that facilitates adjustment of the height of guidance assembly 16 to accommodate different sized patients. In at least one embodiment, height adjustment posts 28 may be threaded and configured to engage with correspondingly threaded interior post receptacles.

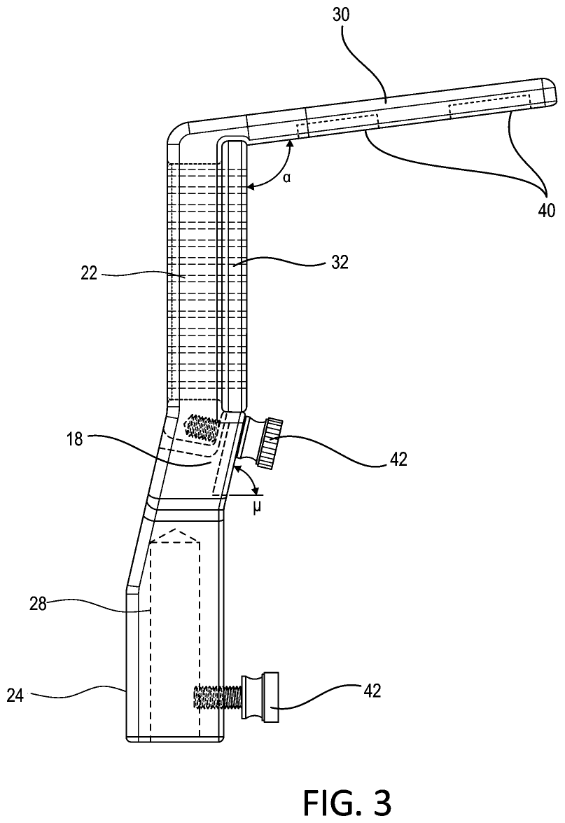

Guidance assembly 16 further includes a top platter 30 that extends from access frame 22. In some embodiments, as illustrated in FIG. 3, for example, top platter 30 forms a platter angle α with access frame 22 of between about 90 degrees and about 120 degrees. Top platter 30 may support the patient's scrotum and penis so as not to obstruct the intervention area

To facilitate instrument guidance, in some embodiments, a guidance plate 32 may be integrally formed with access frame 22 or removably attached or engaged with access frame 22. In some embodiments, guidance plate 32 may be coupled to access frame 22 proximate to the bottom of the access frame and friction fit with top platter 30 at the top of access frame 22. In some embodiments, access frame 22 includes a mounting tab 33 aligned with a corresponding mounting tab that extends from the bottom of access frame 22, as shown in FIG. 2 for example. The mounting tab 33 may be connected to the mounting tab of access frame 22 via a fastener 42 such as a thumb screw. Accordingly, in such embodiments, guidance plate 32 may be readily removed from access frame 22 as desired.

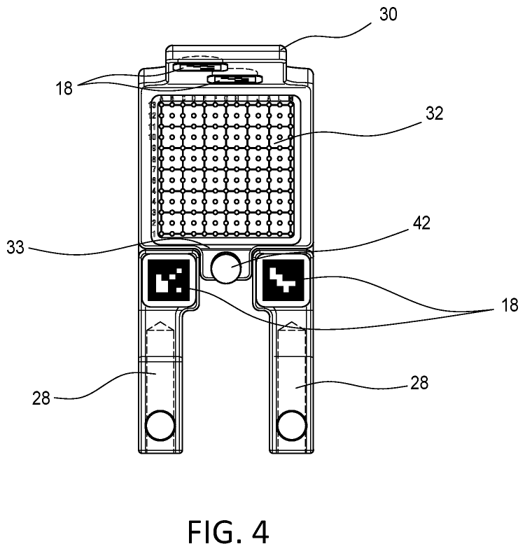

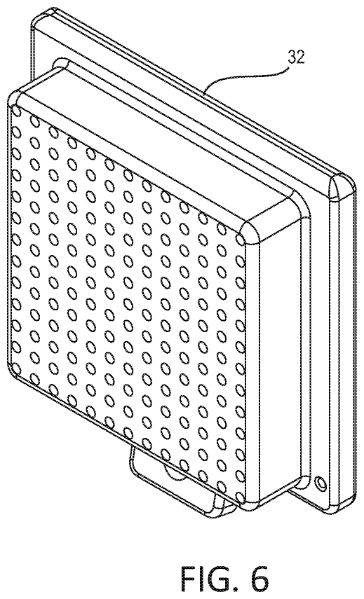

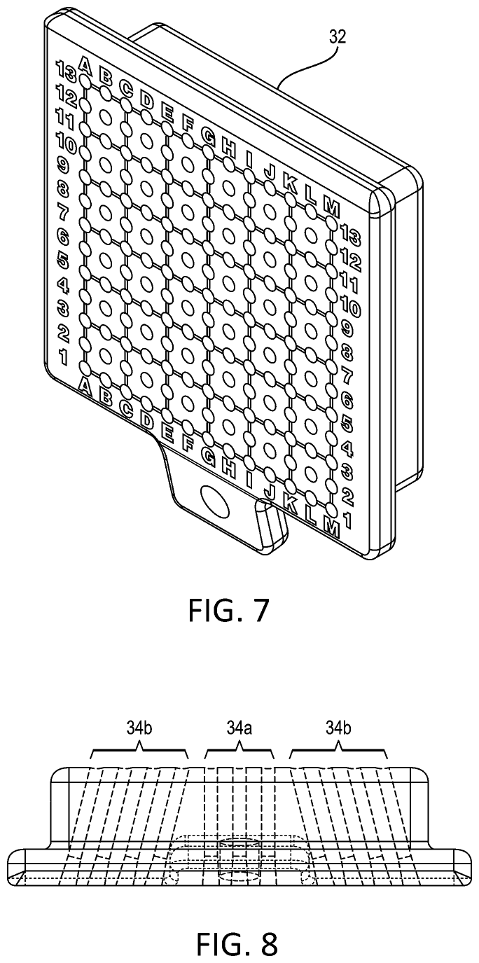

As depicted in FIG. 4, guidance plate 32 may comprise a grid having one or more channels 34. In some embodiments the one or more generally cylindrical channels 34 may have identical angled walls, e.g., orthogonal, and in other embodiments the one or more channels 34 may have different angled walls, e.g., one or more acute and/or one or more obtuse and one or more orthogonal. Accordingly, guidance plate 32 may be easily substituted with another having channels 34 of a different configuration according to the type of instrument used for the intervention and the type of intervention to be performed. For example, a representative guidance plate 32 in accordance with at least one embodiment is illustrated in FIGS. 6-8. There, guidance plate 32 includes first grid pattern having a central set of channels 34a having substantially straight walls and flanking sets of channels 34b having angled walls.

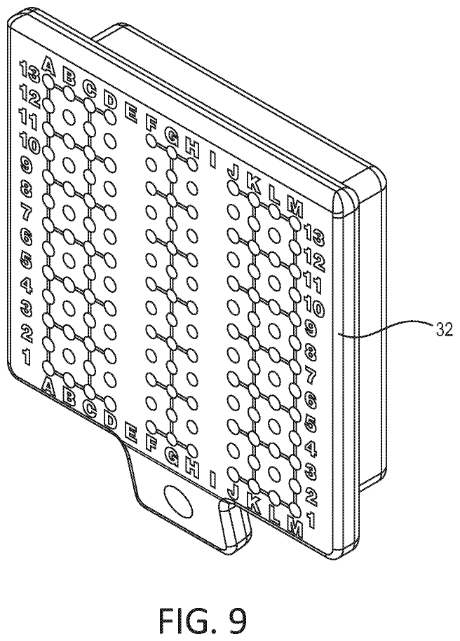

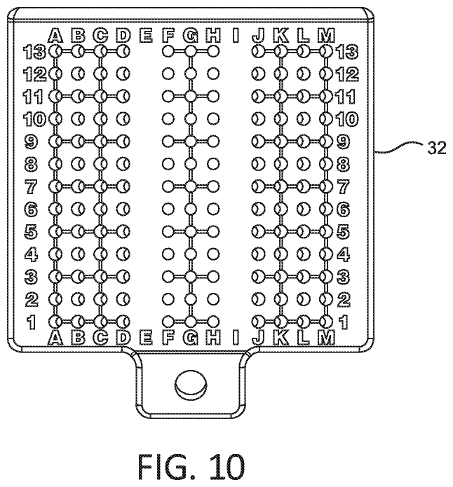

FIGS. 9 and 10 illustrates still another embodiment of guidance plate 32 having a different grid pattern from that shown in FIGS. 6-8.

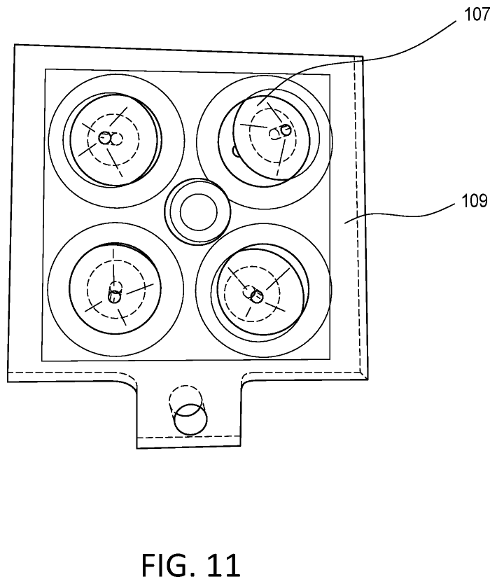

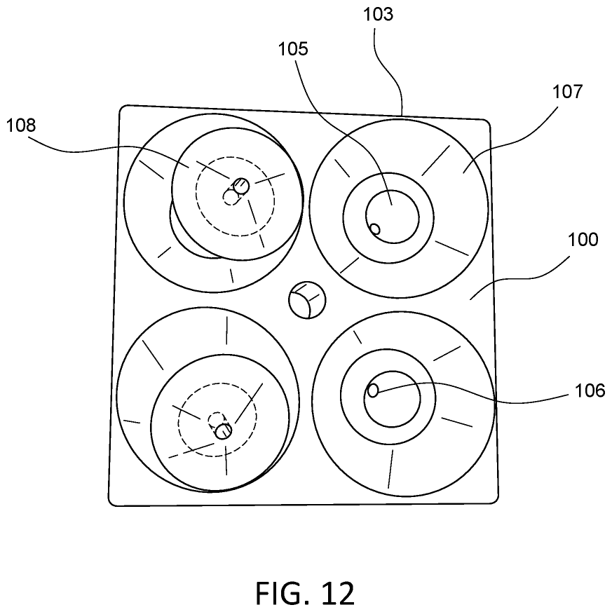

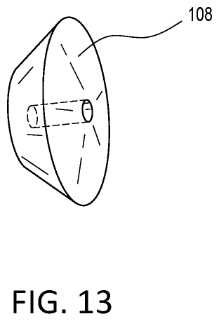

In still another embodiment, guidance plate 32 may comprise a multi-component structure as shown in FIGS. 11-13. With reference to FIG. 12, in one embodiment, guidance plate 32 includes a base 100 having a plurality of telescoping conical channels 103, each including an inner cone 105 and outer cone 107. Each telescoping conical channel 103 includes an aperture 106 of sufficient radius to allow intervention instrument such as a biopsy needle to pass therethrough. Pivot elements 108 are disposed in telescoping conical channels 105 and secured in place by face plate 109. As shown in FIG. 11, pivot elements 108 are solid structures having a through channel of sufficient diameter as to receive the intervention instrument. Face plate 109 may be adjustably secured to base 100 in such a manner as to allow pivot elements 107 move within the telescoping conical channels 103. This freedom of movement allows a user to reposition the intervention instrument while it is in the body and reduces the need for multiple to achieve optimum positioning.

In accordance with another embodiment of the invention, guidance plate 32 may be omitted entirely from access frame 22 leaving a large aperture where guidance plate 32 would otherwise be disposed.

To facilitate image registration, in some embodiments, one or more multi-modal markers 18 may be attached to guidance assembly 16. In one embodiment, first and second multi-modal markers 18 may be attached to access frame 22 and disposed just below upper window 50 as illustrated in FIGS. 3 and 4. In keeping with the invention, the first and second multi-modal markers 18 may be disposed at an acute angle with respect to the plane of base 10 so as to be captured by the camera of optical head 20. This angle is referred to herein as marker angle μ. In some embodiments, marker angle μ may be between about 45° and 90°, preferably between about 75° and 80°. In some embodiments, marker angle μ is about 78°.

The lower surface of top platter 30 may also be provided with one or more multi-modal markers 18. For example, top platter 30 may include one or more marker recesses 40 in which multi-modal markers 18 may be securely mounted and spaced from each other.

Base 10 may be a substantially rigid, plate like structure configured to extend from underneath the buttock of the patient to at least to the mid thigh area and have a width about equal to a standard MRI or CT bed as shown in FIG. 2. In some embodiments, first and second leg separators 12 and 14 are removably attached to base 10 by, e.g., thumb screws 42 and symmetrically disposed about a central longitudinal axis of base 10. Guidance assembly 16 may be positioned between first and second leg separators 12 and 14 such that together, first and second leg separators 12 and 14 and guidance assembly 16 form an “A” frame configuration illustrated for example in FIG. 1.

In accordance with at least one embodiment, optical head 20 is attached to one of first (left) and second (right) separators 12 and 14 and positioned such that optical head 20 has a field of view covering the entire intervention area. In the disclosed embodiments, optical head 20 is attached to an end portion of leg separator 14 to facilitate illumination of the intervention area and localized navigation.

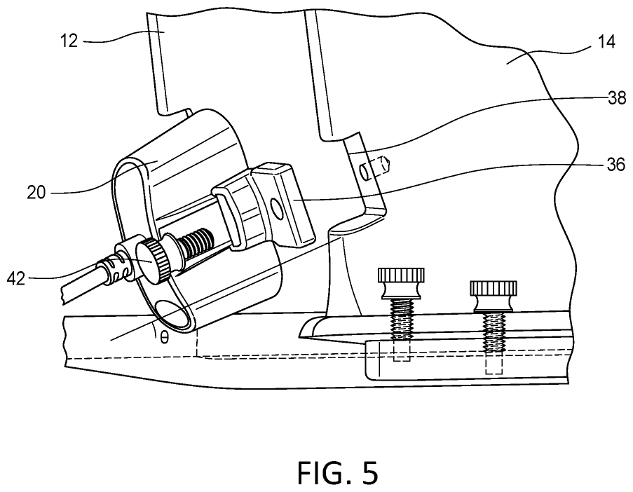

In keeping with the invention, optical head 20 is provided with a mounting bracket 36 that facilitates flush mounting to one of the first and second separators 12 and 14. In some embodiments, such as depicted for example in FIG. 5, mounting bracket 36 is symmetrical, and at least one of the first and second leg separators 12 and 14 is provided with a mounting notch 38 configured to receive mounting bracket 36. Mounting notch 38 may comprise an angled rectangular cutout. Mounting bracket 36 may be lodged in the angled rectangular cutout and engaged therewith by, for example thumb screw 42, such that a pitch angle θ of optical head 20 is fixed and set to provide maximum coverage. Pitch angle θ is the angle formed between the plane of base 10 and a beam axis of optical head 20. This embodiment provides an advantage in that the pitch angle θ is preset to cover a maximum field of view which cannot be altered during the procedure. Since mounting bracket 36 is symmetrical, it can be flipped and attached to leg separator 12. Accordingly, optical head 20 may be optimally positioned for both left and right-handed users. In this embodiment, pitch angle θ is fixed so as to provide a field of view that covers at least 20 cm from access frame 22 and encompasses all multi-modal markers 18 and provides that all multi-modal markers 18 are illuminated by the light source of optical head 20 and that instruments within the field of view are illuminated by the light source.

In accordance with other embodiments, it may be desirable to have an adjustable pitch angle θ. In this embodiment, optical head 20 may be provided, e.g., with a cylindrical joint (not shown) that may be affixed to one of leg separators 12 and 14. Optical head 20 may be rotated about cylindrical joint to its desired position and then locked via thumbscrew 42, for example. Accordingly, a user may adjust pitch angle θ by releasing the thumb screw and rotating optical head 20.

In keeping with the invention, optical head 20 may include a plurality of optical sensors (e.g., cameras) and a plurality of intensity adjustable light sources, (e.g., infrared LEDs and/or visible light LEDs). Optical head 20 may be in communication with a control unit and a display (not shown).

The control unit may receive images and/or data from the plurality of optical sensors and may store MRI and/or ultrasound (US) images and generate and display resent a user interface on the display. The user interface may provide a projection of guidance plate 32 on the MRI/US image. The display may further provide an MRI and US fusion where the two modalities are automatically aligned. The display may further show needle navigation where the location of the tip of the needle, the orientation of the needle, and the projected path of the needle is displayed for the user. The user interface may still further provide a facility to select targets and entry points within the MRI volume and ultrasound images. Point targets or segmented areas of the image may be selected and displayed.

Accordingly, the perineal access tool of the present invention assists the operator in performance of image guided interventions, e.g., prostate interventions, in the MR suite and on the MRI/CT bed. To that end, all components of the perineal access tool may be at least MRI conditional.

For purposes of this disclosure, the term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

1. A perineal access device comprising:
a base;
first and second leg separators removably secured to said base;
a guidance assembly mounted to said base and disposed between said first and second leg separators, the guidance assembly including:
an access frame having an upper window configured to provide access to a patient's perinium and a lower window configured to provide access to the patient's rectum, the access frame being supported by first and second frame legs,
a top platter extending from the access frame, the top platter forming a platter angle with the access frame,
a guidance plate disposed within the access frame, the guidance plate including a grid having one or more channels,
first and second multi-modal markers attached to the first and second frame legs, respectively; and
an optical head attached to one of the first and second leg separators and positioned at a pitch angle to provide a maximum field of view for said optical head.

2. The perineal access device of claim 1 wherein the platter angle is between 90 degrees and 120 degrees.

3. The perineal access device of claim 2 further comprising at least one multi-modal marker mounted on the top platter.

4. The perineal access device of claim 1 wherein each of the first and second frame legs includes an interior post receptacle and further comprising first and second height adjustment posts disposed in the first and second interior post receptacles, respectively.

5. The perineal access device of claim 1 further comprising at least one multi-modal marker attached to said guidance assembly.

6. The perineal access device of claim 5 wherein the at least one multi-modal marker is attached to the access frame and disposed at an acute marker angle.

7. The perineal access device of claim 6 wherein the marker angle is between 45 degrees and 90 degrees.

8. The perineal access device of claim 6 wherein the marker angle is between 75 degrees and 80 degrees.

9. The perineal access device of claim 1 wherein the guidance plate is integrally formed with the upper window.

10. The perineal access device of claim 1 wherein the guidance plate is removably engaged with the upper window.

11. The perineal access device of claim 1 wherein at least one of the one or more channels of the grid has orthogonal walls.

12. The perineal access device of claims 1 and 11 wherein at least one of the one or more channels of the grid has acute angled walls.

13. The perineal access device of claim 1 at least one of the one or more channels of the grid has obtuse angled walls.

14. The perineal access device of claim 1 wherein said optical head includes a plurality of optical sensors and a plurality of adjustable light sources.

15. The perineal access device of claim 1 wherein said optical head is fixedly mounted to one of the first and second leg separators.

16. The perineal access device of claim 1 wherein said guidance assembly is adjustably mounted to said base.

17. The perineal access device of claim 16 including means for adjusting a height of said guidance assembly.

18. A perineal access device comprising:
a base;
first and second leg separators removably secured to said base;
a guidance assembly connected to said base and disposed between said first and second leg separators, the guidance assembly comprising:
an access frame including an upper window configured to provide access to a patient's perinium and a lower window configured to provide access to a patient's rectum; and
an optical head attached to one of the first and second leg separators and positioned at a pitch angle to provide a maximum field of view for said optical head.

19. The perineal access device of claim 18 further comprising at least one multi-modal marker mounted on the top platter.

20. A guidance assembly for a perineal access device comprising:
an access frame having an upper window configured to provide access to a patient's perinium and a lower window configured to provide access to the patient's rectum, the access frame being supported by first and second frame legs,
a top platter extending from the access frame, the top platter forming a platter angle with the access frame,
a guidance plate disposed within the access frame, the guidance plate including a grid having one or more channels,
first and second multi-modal markers attached to the first and second frame legs, respectively.
